(19) United States
(12) Patent Application Publication
(10) Pub. No.: US 2017/0151138 A1
(43) Pub. Date: Jun. 1, 2017

(54) METHOD FOR REDUCING PERSPIRATION AND/OR BODY ODOR USING SPECIFIC ALCOHOLS

(71) Applicant: Henkel AG & Co. KGaA, Duesseldorf (DE)

(72) Inventors: Bernhard Banowski, Duesseldorf (DE); Marcus Claas, Hilden (DE)

(73) Assignee: Henkel AG & Co. KGaA, Duesseldorf (DE)

(21) Appl. No.: 15/365,178

(22) Filed: Nov. 30, 2016

(30) Foreign Application Priority Data
Dec. 1, 2015 (DE) 10 2015 223 846.6

(51) Int. Cl. A61K 8/34; A61Q 19/10; A61K 8/06; A61K 8/04; A61Q 15/00; A61K 8/02

(52) U.S. Cl. CPC A61K 8/345; A61Q 15/00; A61K 8/0229; A61K 8/068; A61K 8/044; A61K 8/062; A61K 8/064; A61Q 19/10

(57) ABSTRACT

A cosmetic method for reducing perspiration of the body and/or for reducing body odor triggered by perspiration is provided herein. In one embodiment, the method includes the step of applying an antiperspirant cosmetic agent (M1) to human skin. The method further includes the step of maintaining the antiperspirant cosmetic agent (M1) on the application site for at least an hour. A packaging unit is also provided herein. In another embodiment, the packaging unit includes, formulated separately from one another, a) at least one first container (C1), containing a cosmetic agent (M1), and b) at least one second container (C2), containing a cosmetic agent (M2).

### CROSS-REFERENCE TO RELATED APPLICATION

This application claims priority to German Patent Application No. 10 2015 223 846.6, filed Dec. 1, 2015, which is incorporated herein by reference in its entirety.

### TECHNICAL FIELD

This disclosure relates to a method for reducing perspiration of the body and/or for reducing body odor triggered by perspiration, in which an antiperspirant cosmetic agent (M1), comprising specific alcohols, is applied to the human skin and remains on the application site for at least one hour. This antiperspirant agent (M1) comprises no more than 1 wt. % antiperspirant aluminum salts and/or aluminum-zirconium salts. The use of the at least one specific alcohol in the method as contemplated herein results in an antiperspirant action or in a reduction of body odor triggered by perspiration.

### BACKGROUND

The present disclosure further relates to the use of at least one specific alcohol for reducing perspiration of the body and/or for reducing body odor triggered by perspiration.

Washing, cleaning, and caring for one's body is a basic human need, and modern industry is continually attempting to satisfy these needs of humans in a variety of ways. The lasting elimination, or at least reduction, of body odor and underarm perspiration is particularly important for daily hygiene. Numerous special deodorizing or antiperspirant body care agents are known in the related art, which were developed for use in body regions that have a high density of sweat glands, in particular in the axilla region. These are formulated in a wide variety of forms of administration, for example as powders, in stick form, as aerosol sprays, pump sprays, liquid and gel-like roll-on applications, creams, gels, and as saturated flexible substrates (deodorant wipes).

The cosmetic antiperspirants used in methods of the related art for reducing perspiration contain at least one antiperspirant compound, in particular in the form of aluminum and/or zirconium halides and/or hydroxy halides. These antiperspirant compounds decrease the secretion of sweat by the body by temporarily constricting and/or clogging the excretory ducts of the sweat glands, whereby the amount of sweat can be reduced by approximately 20 to 60 percent. In addition, due to the antimicrobial action thereof, they prevent initially odorless sweat from degrading into malodorous compounds, thus preventing the development of body odor.

However, the aluminum and/or zirconium halides and/or hydroxy halides present in the agents that are used, in conjunction with the acid pH value of these agents, can result in unpleasant skin reactions in some users. Moreover, the use of the above-mentioned antiperspirant compounds may result in staining on clothing.

A need therefore exists to replace the antiperspirant aluminum and/or zirconium halides and/or hydroxy halides used in method for reducing perspiration with other antiperspirant cosmetic active ingredients. These active ingredients are to exhibit a good antiperspirant action, have good skin tolerability, and be easy to formulate. Moreover, these antiperspirant active ingredients are not to adversely affect the shelf life of the cosmetic agents used.

### BRIEF SUMMARY

A cosmetic method for reducing perspiration of the body and/or for reducing body odor triggered by perspiration is provided herein. In one embodiment, the method includes the step of applying an antiperspirant cosmetic agent (M1) to human skin. The antiperspirant cosmetic agent (M1), in a cosmetically compatible carrier, comprises, based on the total weight of the cosmetic agent (M1), a) about 0.1 to about 5.0 wt. % of at least one alcohol of formulas (I) and/or (II)

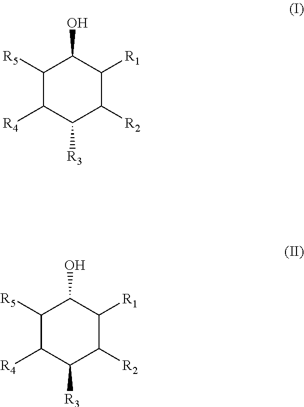

where R₁ to R₅, each independently of one another, denote hydrogen, a linear or branched C₁ to C₁₀ alkyl group or a linear or branched C₂ to C₁₀ alkylene group; and b) no more than 1 wt. % antiperspirant aluminum salts and/or aluminum-zirconium salts. The method further includes the step of maintaining the antiperspirant cosmetic agent (M1) on the application site for at least an hour.

A packaging unit is also provided herein. In another embodiment, the packaging unit includes, formulated separately from one another, a) at least one first container (C1), containing a cosmetic agent (M1), and b) at least one second container (C2), containing a cosmetic agent (M2). The cosmetic agent (M1) includes, in a cosmetically compatible carrier, at least one alcohol of formulas (I) and/or (II)

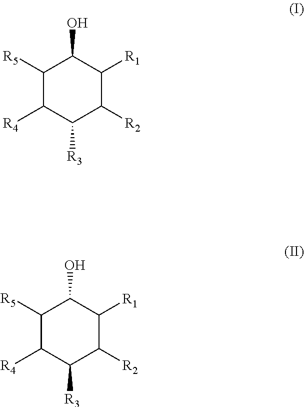

where R₁ to R₅, each independently of one another, denote hydrogen, a linear or branched C₁ to C₁₀ alkyl group or a linear or branched C₂ to C₁₀ alkylene group; and no more than 1 wt. % antiperspirant aluminum salts and/or aluminum-zirconium salts, based on the total weight of the cosmetic agent (M1). The cosmetic agent (M2) includes at least one antiperspirant active ingredient.

### DETAILED DESCRIPTION

The following Detailed Description is merely exemplary in nature and is not intended to limit the various embodiments or the application and uses thereof. Furthermore, there is no intention to be bound by any theory presented in the preceding background or the following detailed description.

It was therefore the object of the present disclosure to provide a method for reducing perspiration of the body and/or for reducing body odor triggered by perspiration which prevents, or at least lessens, the drawbacks of the related art and results in a reliable reduction of underarm perspiration, while offering good skin tolerability.

Surprisingly, it was now found that the use of at least one specific alcohol in cosmetic methods results in an antiperspirant and/or odor-inhibiting action, which is almost comparable to the antiperspirant and odor-inhibiting action achieved by aluminum salts and/or aluminum-zirconium salts used in methods of the related art. No skin irritation whatsoever, however, was observed when these alcohols were used.

The subject matter of the present disclosure is thus a cosmetic method for reducing perspiration of the body and/or for reducing body odor triggered by perspiration, in which an antiperspirant cosmetic agent (M1) is applied to the human skin and remains on the application site for at least one hour, wherein the antiperspirant cosmetic agent (M1), in a cosmetically compatible carrier, comprises, based on the total weight of the cosmetic agent (M1),
a) about 0.1 to about 5.0 wt. % of at least one alcohol of formulas (I) and/or (II)

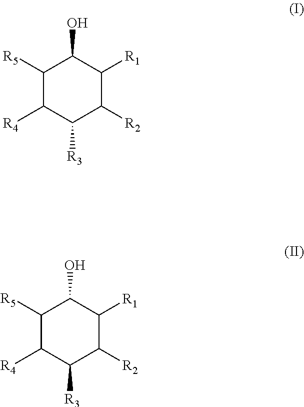

where
R₁ to R₅, each independently of one another, denote hydrogen, a linear or branched C₁ to C₁₀ alkyl group or a linear or branched C₂ to C₁₀ alkylene group; and
b) no more than 1 wt. % antiperspirant aluminum salts and/or aluminum-zirconium salts.

Through the use of the above-described cosmetic agents (M1) in the method as contemplated herein, an outstanding reduction of perspiration, in particular axillary perspiration, is achieved, without skin irritations occurring. Moreover, a reduced development of body odor is observed with the use of the at least one specific alcohol of formulas (I) and/or (II). In this way, an effective reduction of axillary perspiration and/or body odor is ensured even with the use of extremely small amounts or in the absence of antiperspirant aluminum and/or zirconium halides and/or hydroxy halides.

As contemplated herein, the term “antiperspirant” shall be understood to mean the decrease or reduction in perspiration of the body's sweat glands.

Moreover, the term “aluminum salts and/or aluminum-zirconium salts” within the scope of the present disclosure shall be understood to mean in particular aluminum and/or zirconium chlorides, bromides and iodides, and compounds of formulas Al(OH)_{y}X and Zr(OH)_{z}X, wherein X denotes a halide ion in the aforementioned formulas.

Moreover, the term “cosmetic oil” within the meaning of the present disclosure shall be understood to mean an oil that is suitable for cosmetic use and not miscible with water. The cosmetic oil used as contemplated herein involves neither odorous substances nor essential oils.

Moreover, the term “waxes” within the scope of the present disclosure shall be understood to mean substances that are kneadable or solid to brittle-hard at 20° C., have a coarse to microcrystalline structure, and are translucent to colors to opaque, but not vitreous. These substances furthermore melt above 25° C. without decomposing, are easily liquid (low viscosity) just above the melting point, have a highly temperature-dependent consistency and solubility, and can be polished under light pressure.

Finally, the term “fatty acids”, as it is used within the scope of the present disclosure, shall be understood to mean aliphatic carboxylic acids that contain unbranched or branched carbon groups having 4 to 40 carbon atoms. The fatty acids used within the scope of the present disclosure can be both naturally occurring and synthetically produced fatty acids. The fatty acids can moreover be monounsaturated or polyunsaturated.

Unless indicated otherwise, in the present disclosure the wt. % information refers to the total weight of the antiperspirant cosmetic agents (M1) used as contemplated herein, wherein the total amount of all ingredients of the cosmetic agent (M1) is 100 wt. %. Furthermore, unless indicated otherwise, the wt. % information refers to the amount of the particular component in the propellant-free cosmetic agent, so that the amount of possibly present propellant is not considered in the calculation of the total weight of the cosmetic agent.

It is advantageous as contemplated herein that the antiperspirant cosmetic agent (M1) is used to reduce perspiration of the axilla and/or to reduce axillary odor. Preferred methods as contemplated herein are thus characterized in that the antiperspirant cosmetic agent (M1) is applied to the skin of the axilla region.

The antiperspirant cosmetic agents (M1) used as contemplated herein comprise the at least one specific alcohol of formulas (I) and/or (II) in a cosmetic carrier. It is preferred as contemplated herein if this carrier is free of water and ethanol and comprises at least one C₃-C₁₀ diol and/or at least one cosmetic oil that is liquid at 20° C. and 1,013 hPa.

As contemplated herein, an anhydrous carrier shall be understood to mean a carrier that comprises less than 5.0 wt. %, especially less than 2.0 wt. %, and in particular 0 wt. % free water, based on the total weight of the antiperspirant cosmetic agent (M1). “Free water” within the meaning of the present disclosure shall be understood to mean water that is different from constitutional water, hydration water or similarly molecularly bound water present in the components used. Furthermore, as contemplated herein, an ethanol-free carrier shall be understood to mean a carrier that comprises less than 5.0 wt. %, especially less than 2.0 wt. %, and in particular 0 wt. % ethanol, based on the total weight of the antiperspirant cosmetic agent (M1). Preferred agents (M1) as contemplated herein are thus characterized in that the cosmetic carrier comprises 0 wt. % water and/or ethanol.

Carriers that are preferably used as contemplated herein are cosmetic oils that are liquid at 20° C. and 1,013 hPa. The cosmetic oils that are liquid at 20° C. and 1,013 hPa are selected from the group consisting of (i) volatile cyclic silicone oils, in particular cyclic and linear silicone oils; (ii) volatile non-silicone oils, in particular liquid paraffin oils and isoparaffin oils; (iii) non-volatile silicone oils; (iv) non-volatile non-silicone oils; and (v) the mixtures thereof.

The term “volatile oil” as contemplated herein refers to oils that, at 20° C. and an ambient pressure of 1,013 hPa, have a vapor pressure of about 2.66 Pa to about 40,000 Pa (0.02 to 300 mm Hg), especially of about 10 to about 12,000 Pa (0.1 to 90 mm Hg), more preferably of about 13 to about 3,000 Pa (0.1 to 23 mm Hg), and in particular of about 15 to about 500 Pa (0.1 to 4 mm Hg).

Moreover, the term “non-volatile oils” within the meaning of the present disclosure shall be understood to mean oils that, at 20° C. and an ambient pressure of 1,013 hPa, have a vapor pressure of less than 2.66 Pa (0.02 mm Hg).

It may be preferred as contemplated herein to use mixtures of volatile silicone oils and volatile non-silicone oils as the carrier, since in this way a dryer skin sensation is achieved. It may moreover be preferred within the scope of the present disclosure to use a non-volatile silicone oil and/or a non-volatile non-silicone oil as the carrier so as to mask insoluble components, such as talcum or ingredients dried on the skin.

Particularly preferred as contemplated herein is the use of mixtures of non-volatile and volatile cosmetic oils, since in this way parameters such as skin sensation, visibility of the residue, and stability of the antiperspirant cosmetic agent (M1) used as contemplated herein can be set, and the agent can thus be better adapted to the needs of the consumers.

The volatile and non-volatile silicone oils and volatile and non-volatile non-silicone oils that can be used within the scope of the present disclosure are disclosed in the unexamined patent applications DE 10 2010 063 250 A1 and DE 10 2012 222 692 A1, for example.

The cosmetic oil liquid at 20° C. and 1,013 hPa is preferably used in a total amount of about 1.0 to about 98 wt. %, especially about 2.0 to about 85 wt. %, preferably about 4.0 to about 75 wt. %, more preferably about 6.0 to about 70 wt. %, still more preferably about 8.0 to about 60 wt. %, and in particular about 8.0 to about 20 wt. %, based on the total weight of the antiperspirant cosmetic agent (M1).

Within the scope of the present disclosure, a C₃-C₁₂ diol is preferably used as the cosmetic carrier so as to ensure sufficient solubility of the at least one alcohol of formulas (I) and/or (II). Preferred cosmetic agents (M1) as contemplated herein are thus characterized by comprising at least one C₃-C₁₂ diol from the group consisting of 1,2-propanediol, 1,3-propanediol, 2-methylpropane-1,3-diol, 1,2-butanediol, 1,3-butanediol, 1,4-butanediol, 1,2-pentanediol, 1,3-pentanediol, 1,5-pentanediol, 2,4-pentanediol, 2-methylpentane-2,4-diol, 3-methylpentane-1,3-diol, 3-methylpentane-1,5-diol, 1,2-hexanediol, 1,6-hexanediol, 2-ethyl hexanediol, 1,2-octanediol, dodecanediol, dipropylene glycol and the mixtures thereof, and in particular 1,2-propanediol and/or dodecanediol, as the cosmetically compatible carrier.

The cosmetic agent (M1) used as contemplated herein comprises at least one alcohol of formulas (I) and/or (II) in certain quantity ranges as the first essential component. This alcohol is essential for the antiperspirant and/or odor-inhibiting action of the method as contemplated herein which is achieved.

It has proven to be advantageous within the scope of the present disclosure to use alcohols of formulas (I) and/or (II) in which the groups R₁, R₂, R₄ and R₅ denote certain substituents. It is thus preferred as contemplated herein if the groups R₁, R₂, R₄ and R₅ in formulas (I) and/or (II), each independently of one another, denote hydrogen.

It has furthermore proven to be advantageous within the scope of the present disclosure if the at least one alcohol of formulas (I) and/or (II) comprises a branched alkyl group having at least 3 carbon atoms as group R₃. Cosmetic agents (M1) that are preferably used are thus characterized in that the group R₃ in formulas (I) and/or (II) denotes a branched C₃ to C₈ alkyl group, preferably a branched C₃ to C₇ alkyl group, and in particular a branched C₃ to C₅ alkyl group.

Particularly good results are achieved if both the groups R₁, R₂, R₄ and R₅ and the group R₃ in formulas (I) and/or (II) denote certain substituents. It is thus particularly preferred as contemplated herein if the antiperspirant cosmetic agent (M1) comprises at least one alcohol of formulas (Ia) and/or (IIa)

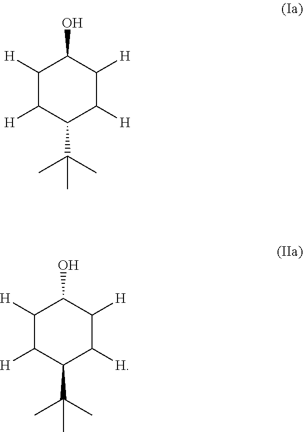

The use of the above-described alcohols of formulas (Ia) and/or (IIa) in the method as contemplated herein results in a particularly high reduction of perspiration and of body odor triggered by perspiration. The above-mentioned alcohols are known by the name trans-4-tert-Butylcyclohexanol (CAS number: 21862-63-5) and can be obtained, for example, by reducing p-tert-Butylcyclohexanone with lithium aluminum hydride and aluminum chloride and recrystallization with petrol ether.

Within the scope of the present disclosure, it is advantageous to use the at least one alcohol (I) and/or (II), and in particular (Ia) and/or (IIa), in the cosmetic agents (M1) used as contemplated herein in certain quantity ranges. Preferred methods as contemplated herein are thus characterized in that the cosmetic agent (M1), based on the total weight thereof, comprises about 0.1 to about 5.0 wt. %, especially about 0.6 to about 3.0 wt. %, preferably about 0.7 to about 2.0 wt. %, and in particular about 0.8 to about 1.5 wt. % of at least one alcohol of formulas (I) and/or (II), and in particular of formulas (Ia) and/or (Ib).

The antiperspirant and/or odor-inhibiting action achieved by way of the method as contemplated herein is preferably achieved solely by using the at least one alcohol of formulas (I) and/or (II), and in particular (Ia) and/or (IIa). It is thus advantageous within the scope of the present disclosure if the cosmetic agent (M1) comprises 0 wt. % of antiperspirant aluminum salts and/or aluminum-zirconium salts, based on the total weight of the cosmetic agent (M1). Cosmetic agents (M1) that are particularly preferably used as contemplated herein thus do not contain any antiperspirant salts of aluminum salts and/or aluminum-zirconium whatsoever. In particular, preferably none of the antiperspirant salts of aluminum salts and/or aluminum-zirconium mentioned below are present:
(i) water-soluble astringent inorganic salts of aluminum, in particular aluminum chlorohydrate, aluminum sesquichlorohydrate, aluminum dichlorohydrate, aluminum hydroxide, potassium aluminum sulfate, aluminum bromohydrate, aluminum chloride, aluminum sulfate;
(ii) water-soluble astringent organic salts of aluminum, in particular aluminum chlorohydrex propylene glycol, aluminum chlorohydrex polyethylene glycol, aluminum propylene glycol complexes, aluminum sesquichlorohydrex propylene glycol, aluminum sesquichlorohydrex polyethylene glycol, aluminum propylene glycol dichlorohydrex, aluminum polyethylene glycol dichlorohydrex, aluminum undecylenoyl collagen amino acid, sodium aluminum lactate, sodium aluminum chlorohydroxy lactate, aluminum lipoamino acids, aluminum lactate, aluminum chlorohydroxy allantoinate, sodium aluminum chlorohydroxy lactate;
(iii) water-soluble astringent inorganic aluminum-zirconium salts, in particular aluminum-zirconium trichlorohydrate, aluminum-zirconium tetrachlorohydrate, aluminum-zirconium pentachlorohydrate, aluminum-zirconium octachlorohydrate;
(iv) water-soluble astringent organic aluminum-zirconium salts, in particular aluminum-zirconium propylene glycol complexes, aluminum-zirconium trichlorohydrex glycine, aluminum-zirconium tetrachlorohydrex glycine, aluminum-zirconium pentachlorohydrex glycine, aluminum-zirconium octachlorohydrex glycine; and
(v) the mixtures thereof.

The antiperspirant cosmetic agents (M1) used in the method as contemplated herein preferably have a certain pH value. Within this range, a stable formulation of the antiperspirant cosmetic agents (M1) used as contemplated herein is possible, without resulting in undesirable interactions between the ingredients. Moreover, no skin irritation occurs with the use of these agents at these pH values. It is thus advantageous if the antiperspirant cosmetic agent (M1) has a pH value of about pH 2 to about pH 10. The desired pH value can be set by using acids and bases that are known to a person skilled in the art and customary in antiperspirant cosmetic agents.

The antiperspirant cosmetic agent (M1) used in the method as contemplated herein may include further substances, in addition to the above-described ingredients.

As contemplated herein, the antiperspirant cosmetic agent (M1) preferably additionally comprises at least one further auxiliary substance, selected from the group consisting of (i) emulsifiers and/or surfactants; (ii) thickeners; (iii) chelating agents; (iv) deodorant active ingredients; (v) polyethylene glycols; (vi) skin-cooling active ingredients; (vii) pH-setting agents; (viii) skin care active ingredients, such as moisturizers, skin-soothing substances, skin-lightening substances, skin-smoothing substances; (ix) waxes); (x) preservatives; and (xi) the mixtures thereof.

Preferably suited emulsifiers and surfactants as contemplated herein are selected from anionic, cationic, non-ionic, amphoteric, in particular ampholytic and zwitterionic, emulsifiers and surfactants. Surfactants are amphiphilic (bifunctional) compounds, which are composed of at least one hydrophobic molecule part and at least one hydrophilic molecule part. The hydrophobic group is preferably a hydrocarbon chain having 8 to 28 carbon atoms, which can be saturated or unsaturated, linear or branched. This C₈ to C₂₈ alkyl chain is particularly preferably linear.

For thickening the antiperspirant cosmetic agents (M1) used as contemplated herein preferably substances are used that are selected from cellulose ethers, xanthan gum, sclerotium gum, succinoglycans, polygalactomannan gums, pectins, agar, caragheen (carrageenan), tragacanth, gum arabic, karaya gum, tara gum, gellan gum, gelatin, propylene glycol alginate, alginic acids and the salts thereof, polyvinylpyrrolidones, polyvinyl alcohols, polyacrylamides, physically (such as by way of pre-gelatinization) and/or chemically modified starches, acrylic acid/acrylate copolymers, acrylic acid/acrylamide copolymers, acrylic acid/vinlypyrrolidone copolymers, acrylic acid/vinylformamide copolymers and polyacrylates. The use of cellulose ethers, such as carboxymethyl celluloses, as thickeners is particularly preferred. Particularly preferred thickeners are furthermore selected from carbomers. Carbomers are thickening cross-linked polymers of acrylic acid, methacrylic acid, and the salts. The cross-linking can take place by way of polyfunctional compounds such as polyalkylene ether of polysaccharides or polyalcohols, for example allyl ether of sucrose, allyl ether of pentaerythritol, allyl ether of propylene. Homopolymers of acrylic acid or the salts thereof, which are cross-linked with an allyl ether of pentaerythritol, an allyl ether of sucrose, or an allyl ether of propylene, are preferred within the scope of the present disclosure. One thickener that can be used within the scope of the present disclosure is a copolymer of C₁₀₋₃₀ alkyl acrylate, acrylic acid, methacrylic acid, and the esters thereof, which is cross-linked with an allyl ether of sucrose or an allyl ether of pentaerythritol. Carbomer-based thickeners are the products available by the trade name Carbopol® (BF Goodrich, Ohio, USA), such as Carbopol 934, Carbopol 940, Carbopol 941, Carbopol 971, Carbopol 974, Carbopol EZ2, Carbopol ETD 2001, Carbopol ETD 2020, Carbopol ETD 2050, Carbopol ultrez 10, Carbopol ultrez 20, or Carbopol ultrez 21.

Furthermore, lipophilic thickeners can be used to thicken the antiperspirant cosmetic agents (M1) used as contemplated herein. Preferred lipophilic thickeners as contemplated herein are selected from hydrophobized clay minerals, bentonites, hectorites, fumed silica, and the derivatives thereof.

As an additional auxiliary substance, the antiperspirant cosmetic agents (M1) used as contemplated herein can comprise at least one chelating agent in a total amount of 0.01 to 3.0 wt. %, especially of 0.02 to 1.0 wt. %, and in particular of 0.05 to 0.1 wt. %, based on the total weight of the antiperspirant agent (M1). Within the scope of the present disclosure, preferred chelating agents are selected from the group consisting of R-alanine diacetic acid, cyclodextrin, diethylenetriamine pentamethylene phosphonic acid, sodium-, potassium-, calcium disodium-, ammonium- and triethanolamine salts of ethylenediaminetetraacetic acid (EDTA), etidronic acid, hydroxyethyl ethylenediaminetriacetic acid (HEDTA) and the sodium salts thereof, sodium salts of nitrilotriacetic acid (NTA), diethylene triamine pentaacetic acid, phytinic acid, hydroxypropyl cyclodextrin, methyl cyclodextrin, pentasodium aminotrimethylene phosphonate, pentasodium ethylenediamine tetramethylene phosphonate, pentasodium diethylene triamine pentaacetate, pentasodium triphosphate, potassium EDTMP, sodium EDTMP, sodium dihydroxyethyl glycinate, sodium phytate, sodium polydimethylglycinophenol sulfonate, tetrahydroxyethyl ethylenediamine, tetrahydroxypropyl ethylenediamine, tetrapotassium etidronate, tetrasodium etidronate, tetrasodium iminodisuccinate, trisodium ethylenediamine disuccinate, tetrasodium-N,N-bis(carboxymethyl)glutamate, tetrasodium-DL-Alanine-N,N-diacetate and desferrioxamine.

The deodorizing action of the antiperspirant cosmetic agents (M1) used as contemplated herein can be further increased if additionally at least one deodorant active ingredient having an antibacterial and/or bacteriostatic and/or enzyme-inhibiting and/or odor-neutralizing and/or odor-absorbing action is present in a total amount of about 0.0001 to about 40 wt. %, especially about 0.2 to about 20 wt. %, preferably about 1 to about 15 wt. %, and in particular about 1.5 to about 5.0 wt. %, based on the total weight of the antiperspirant cosmetic agent (M1). If ethanol is used in the agents used as contemplated herein, this is not considered an active deodorant ingredient within the scope of the present disclosure, but a component of the carrier.

Preferred antiperspirant cosmetic agents (M1) used as contemplated herein can furthermore contain at least one water-soluble polyethylene glycol having 3 to 50 ethylene oxide units.

Moreover, the antiperspirant cosmetic agents (M1) used as contemplated herein can also comprise at least one skin-cooling active ingredient. Suitable skin-cooling active ingredients as contemplated herein are, for example, menthol, isopulegol and menthol derivatives, such as menthyl lactate, menthyl glycolate, menthyl ethyl oxamate, menthyl pyrrolidone carboxylic acid, menthyl methyl ether, menthoxypropanediol, menthone glycerin acetal (9-methyl-6-(1-methylethyl)-1,4-dioxaspiro(4.5)decane-2-methanol), monomenthyl succinate, 2-hydroxymethyl-3,5,5-trimethylcyclohexanol, and 5-methyl-2-(1-methylethyl)cyclohexyl-N-ethyloxamate. Menthol, isopulegol, menthyl lactate, menthoxypropanediol, menthylpyrrolidone carboxylic acid and 5-methyl-2-(1-methylethyl)cyclohexyl-N-ethyloxamate and mixtures of these substances, in particular mixtures of menthol and menthyl lactate, menthol, menthol glycolate and menthyl lactate, menthol and menthoxypropanediol, or menthol and isopulegol, are preferred as skin-cooling active ingredients.

Preferred pH-setting agents as contemplated herein are acids and/or alkalizing agents and/or buffers. As contemplated herein, preferably inorganic acids (such as hydrochloric acid, sulfuric acid or phosphoric acid) or organic acids (such as citric acid, tartaric acid or malic acid) are the acids used. Alkalizing agents that can be used as contemplated herein are preferably selected from the group consisting of ammonia, basic amino acids, alkali hydroxides, alkaline earth hydroxides, carbonates and hydrogen carbonates, alkanolamines such as amino-2-methyl-1-propanol, monoethanolamine, triethanolamine, diethanolamine and triisopropanolamine, alkali metal metasilicates, urea, morpholine, N-methylglucamine, imidazole. Preferred alkali metal ions are lithium, sodium, potassium, and in particular sodium or potassium. Suitable buffer systems within the scope of the present disclosure are in particular carbonic acid/bicarbonate buffers, carbonic acid/silicate buffers, acetic acid/acetate buffers, ammonia buffers, citric acid or citrate buffers, buffers based on Tris(hydroxymethyl)aminomethane, buffers based on 4-(2-hydroxyethyl)-1-piperazineethanesulfonic acid, buffers based on 4-(2-hydroxyethyl)-piperazine-1-propanesulfonic acid, buffers based on 2-(N-morpholino)ethanesulfonic acid and barbital/acetate buffers. The selection of the appropriate buffer system depends on the desired pH value of the antiperspirant cosmetic agents (M1) as contemplated herein.

Furthermore, the antiperspirant cosmetic agents (M1) used as contemplated herein can comprise at least one wax. This wax is preferably selected from the group consisting of (i) fatty acid glycerol monoesters, diesters and triesters; (ii) Butyrospermum Parkii (shea butter); (iii) esters of saturated, monohydric C₈₋₁₈ alcohols with saturated C₁₂₋₁₈ monocarboxylic acids; (iv) linear, primary C_{12-C24} alkanols; (v) esters of a saturated, monohydric C₁₆₋₆₀ alkanol and a saturated C_{8-C36} monocarboxylic acid; (vi) glycerol triesters of saturated linear C₁₂₋₃₀ carboxylic acids, which may be hydroxylated, such as glycerol esters of hydrogenated vegetable oils; (vii) natural plant-based waxes; (viii) animal waxes; (ix) synthetic waxes; and (x) the mixtures thereof.

The wax is preferably used in a total amount of about 0.01 to about 60 wt. %, especially about 3.0 to about 40 wt. %, preferably about 5.0 to about 30 wt. %, and in particular about 6.0 to about 25 wt. %, based on the total weight of the antiperspirant cosmetic agent (M1).

As contemplated herein, it is furthermore preferred if the antiperspirant cosmetic agent (M1) used as contemplated herein additionally comprises at least one preservative. Preferred preservatives as contemplated herein are formaldehyde cleavers, iodopropinyl butylcarbamates, parabens, phenoxyethanol, ethanol, benzoic acid and the salts thereof, dibromodicyanobutane, 2-bromo-2-nitropropane-1,3-diol, imidazolidinyl urea, 5-chloro-2-methyl-4-isothiazolin-3-one, 2-chloroacetamide, benzalkonium chloride, benzyl alcohol, salicylic acid and salicylates. Further preservatives that can be used within the scope of the present disclosure are substances listed in Attachment 6 of the German Cosmetics Regulation as well as cosmetic raw materials having preserving properties or raw materials that support or enhance the preserving action of the above-mentioned preservatives. The preservatives are preferably present in a total amount of about 0.01 to about 10 wt. %, especially about 0.1 to about 7.0 wt. %, preferably about 0.2 to about 5.0 wt. %, and in particular about 0.3 to about 2.0 wt. %, based on the total weight of the antiperspirant cosmetic agent (M1).

It is preferred within the scope of the present disclosure if the antiperspirant cosmetic agent (M1) used as contemplated herein is present in the form of an emulsion. This may be in particular a sprayable emulsion, which can be sprayed by way of a propellant. If the antiperspirant cosmetic agents (M1) used as contemplated herein contain a propellant, this is preferably present in a total amount of about 1 to about 98 wt. %, especially about 20 to about 90 wt. %, preferably about 30 to about 85 wt. %, and in particular about 40 to about 75 wt. %, based on the total weight of the antiperspirant cosmetic agent (M1). Preferred propellants (propellant gases) are propane, propene, n-butane, iso-butane, iso-butene, n-pentane, pentene, iso-pentane, iso-pentene, methane, ethane, dimethylether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropene, and more particularly both individually and in the mixtures thereof. It is also possible to advantageously use hydrophilic propellants, such as carbon dioxide, within the meaning of the present disclosure if the proportion of hydrophilic gases is selected to be low, and lipophilic propellant (such as propane/butane) is present in excess. Propane, n-butane, iso-butane and mixtures of these propellants are particularly preferred. It has been shown that the use of n-butane as the sole propellant may be particularly preferred as contemplated herein.

The antiperspirant cosmetic agent (M1) used as contemplated herein can be applied using different methods. According to a preferred embodiment, the antiperspirant cosmetic agent (M1) used as contemplated herein is formulated as a spray application. The spray application is carried out using a spraying device, which in a container contains a filling composed of the antiperspirant cosmetic agent (M1) used as contemplated herein in liquid, viscous-flowable, suspension or powder form. As described above, the filling can be pressurized by a propellant (pressurized cans, pressurized containers, aerosol dispensers), or it can be a pump atomizer that contains no propellant gas and is to be operated mechanically (pump sprays, squeeze bottle). The atomization of the antiperspirant cosmetic agent (M1) used in the method as contemplated herein may take place physically, mechanically or electromechanically, for example by way of piezo effects or electric pumps.

The antiperspirant cosmetic agent (M1) used as contemplated herein can furthermore preferably formulated as a stick, soft solid, cream, gel, roll-on, or loose or compact powder. The formulation of the antiperspirant cosmetic agents (M1) used as contemplated herein in a particular form of administration, such as an antiperspirant roll-on or an antiperspirant stick or an antiperspirant gel, is preferably dependent on the requirements of the intended purpose. Depending on the intended purpose, the antiperspirant cosmetic agents (M1) used as contemplated herein can thus be present in solid, semi-solid, liquid, disperse, emulsified, suspended, gel-like, multi-phase or powder form. The term “liquid” within the meaning of the present disclosure also covers any type of solid dispersions in liquids. Furthermore, multi-phase antiperspirant cosmetic agents (M1) used as contemplated herein within the meaning of the present disclosure shall be understood to mean agents which contain at least two different phases having a phase separation and in which the phases may be disposed horizontally, which is to say on top of each other, or vertically, which is to say next to each other. The application can take place by way of a roller ball applicator, a pump atomizer or by way of a solid stick, for example.

However, it may likewise be preferred within the scope of the present disclosure for the antiperspirant cosmetic agent (M1) to be present on and/or in a disposable substrate, selected from the group consisting of wipes, pads and puffs. Moist wipes, which is to say moist wipes that are prefabricated for the user, preferably packaged individually, as they are well known from the field of glass cleaning or the field of moist toilet paper, for example, are particularly preferred. Such moist wipes, which advantageously may also contain preservatives, are impregnated with an antiperspirant cosmetic agent (M1) used as contemplated herein, or have the same applied thereto, and are preferably individually packaged. Preferred substrate materials are selected from porous planar wipes. These wipes include wipes made of woven and non-woven synthetic and natural fibers, felt, paper or foam, such as hydrophilic polyurethane foam. Preferred deodorizing or antiperspirant substrates as contemplated herein can be obtained by saturation or impregnation, or else by melting an antiperspirant cosmetic agent (M1) used as contemplated herein onto a substrate.

The following tables list particularly preferred embodiments AF 1 to AF 16 of the cosmetic agents (M1) used in the method as contemplated herein (all information is in wt. %, unless indicated otherwise).

| | AF 1 | AF 2 | AF 3 | AF 4 |
|---|---|---|---|---|
| Alcohol of formulas (I) and/or | 0.1-4.0 | 0.6-3.0 | 0.7-2.0 | 0.8-1.5 |
| (II) ¹⁾ | | | | |
| Aluminum salt and/or aluminum- | 0-1.0 | 0-0.5 | 0-0.2 | 0 |
| zirconium salt | | | | |
| Cosmetic carrier ²⁾ | to make | to make | to make | to make |
| | 100 | 100 | 100 | 100 |
| | AF 5 | AF 6 | AF 7 | AF 8 |
| Alcohol of formulas (I) and/or | 0.1-4.0 | 0.6-3.0 | 0.7-2.0 | 0.8-1.5 |
| (II) ³⁾ | | | | |
| Aluminum salt and/or aluminum- | 0-1.0 | 0-0.5 | 0-0.2 | 0 |
| zirconium salt | | | | |
| Cosmetic carrier ²⁾ | to make | to make | to make | to make |
| | 100 | 100 | 100 | 100 |
| | AF 9 | AF 10 | AF 11 | AF 12 |
| Alcohol of formulas (I) and/or | 0.1-4.0 | 0.6-3.0 | 0.7-2.0 | 0.8-1.5 |
| (II) ⁴⁾ | | | | |
| Aluminum salt and/or aluminum- | 0-1.0 | 0-0.5 | 0-0.2 | 0 |
| zirconium salt | | | | |
| Cosmetic carrier ²⁾ | to make | to make | to make | to make |
| | 100 | 100 | 100 | 100 |
| | AF 13 | AF 14 | AF 15 | AF 16 |
| Alcohol of formulas (Ia) and/or | 0.1-4.0 | 0.6-3.0 | 0.7-2.0 | 0.8-1.5 |
| (IIa) | | | | |
| Aluminum salt and/or aluminum- | 0-1.0 | 0-0.5 | 0-0.2 | 0 |
| zirconium salt | | | | |
| Cosmetic carrier ²⁾ | to make | to make | to make | to make |
| | 100 | 100 | 100 | 100 |

¹⁾ R₁ to R₅, each independently of one another, denote hydrogen, a linear or branched C₁ to C₁₀ alkyl group or a linear or branched C₂ to C₁₀ alkylene group,
²⁾ carrier selected from the group consisting of anhydrous carriers, aqueous carriers or aqueous-alcoholic carriers,
³⁾ R₁, R₂, R₄ and R₅, each independently of one another, denote hydrogen,
⁵⁾ R₃ denotes a branched C₃ to C₈ alkyl group, preferably a branched C₃ to C₇ alkyl group, and in particular a branched C₃ to C₅ alkyl group.

The use of the antiperspirant cosmetic agents of the embodiments AF1 to AF16 in the method as contemplated herein results in an outstanding antiperspirant action. An outstanding antiperspirant action is in particular also achieved in the absence of aluminum salts and/or aluminum-zirconium salts. The cosmetic agents of embodiments AF1 to AF16 furthermore have an outstanding skin tolerability.

It may also be provided within the scope of the present disclosure to apply a further cosmetic agent (M2), which comprises at least one antiperspirant active ingredient. The agents (M1) and (M2) may be applied consecutively in any arbitrary sequence or simultaneously. It may be provided, for example, that first the cosmetic agent (M1) is applied, and thereafter the cosmetic agent (M2). However, it is also possible to first apply the cosmetic agent (M2), and thereafter the cosmetic agent (M1). In addition, the cosmetic agent (M1) and the cosmetic agent (M2) may be applied simultaneously. The time period between the applications of the two agents (M1) and (M2) is preferably from 0 seconds (simultaneous application) to 24 hours.

It is thus preferred within the scope of the present disclosure if a further cosmetic agent (M2), comprising at least one antiperspirant active ingredient in a cosmetically compatible carrier, is applied to the skin. The cosmetic agent (M2) is then different from the cosmetic agent (M1). In particular, the antiperspirant active ingredient in the agent (M2) does not involve the above-described alcohols of formulas (I), (Ia) and (IIa). Preferred antiperspirant active ingredients are the above-described antiperspirant aluminum salts and/or aluminum-zirconium salts. As a result of the additional use of the cosmetic agent (M2), the antiperspirant action of the method as contemplated herein may be improved yet again.

If a further cosmetic agent (M2) is to be used in the method as contemplated herein, it is advantageous to store each of the individual cosmetic agents (M1) and (M2) in separate containers.

A further subject matter of the present disclosure is thus a packaging unit (kit of parts), comprising, formulated separately from one another,
a) at least one first container (C1), containing a cosmetic agent (M1) comprising, in a cosmetically compatible carrier, at least one alcohol of formulas (I) and/or (II)

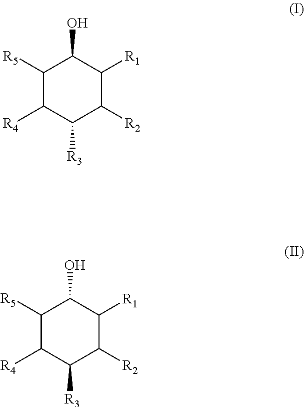

where
R₁ to R₅, each independently of one another, denote hydrogen, a linear or branched C₁ to C₁₀ alkyl group or a linear or branched C₂ to C₁₀ alkylene group; and
and no more than 1 wt. % antiperspirant aluminum salts and/or aluminum-zirconium salts, based on the total weight of the cosmetic agent (M1), and
b) at least one second container (C2), containing a cosmetic agent (M2) comprising at least one antiperspirant active ingredient.

As contemplated herein, the term “antiperspirant active ingredient” shall be understood to mean active ingredients that decrease or reduce the perspiration of the body's sweat glands, wherein, however, the alcohols of formulas (I), (II), (Ia) and (IIa) present in the agent (M1) are not covered by these active ingredients. Within the scope of the present disclosure, the antiperspirant aluminum salts and/or aluminum-zirconium salts listed in connection with the first subject matter of the disclosure are preferably used as the antiperspirant active ingredient in the cosmetic agent (M2).

Within the scope of this embodiment, it is particularly advantageous if the cosmetic agent (M1) comprises at least one alcohol of formulas (Ia) and/or (IIa)

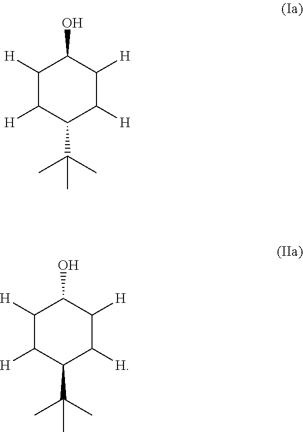

What was said above with respect to the cosmetic agents (M1) used in the method as contemplated herein applies, mutatis mutandis, with respect to cosmetic agent (M1) in the container (C1).

Finally, a further subject matter of the present disclosure is the use of at least one alcohol of formulas (I) and/or (II)

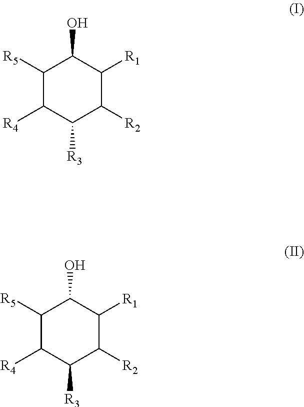

where
R₁ to R₅, each independently of one another, denote hydrogen, a linear or branched C₁ to C₁₀ alkyl group or a linear or branched C₂ to C₁₀ alkylene group; and
   for reducing perspiration of the body and/or for reducing body odor triggered by perspiration.

Within the scope of this embodiment, it is particularly advantageous if at least one alcohol of formulas (Ia) and/or (IIa)

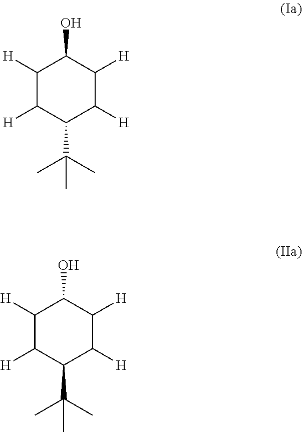

is used.

What was said above with respect to the method as contemplated herein and with respect to the packaging unit as contemplated herein applies, mutatis mutandis, with respect to the alcohol of formulas (I) and/or (II) that is used as contemplated herein.

The present disclosure is outlined in particular by the following items:

The following examples describe the present disclosure in more detail, without limiting it to these examples.

### EXAMPLES

### 1. In-Vivo Test on the Antiperspirant Action

To ascertain the antiperspirant action, an antiperspirant study was conducted on the backs of 17 female subjects. The following antiperspirant agents were used for this purpose:

| Antiperspirant agent | No. |
|---|---|
| Solution of 1,2-propanediol | V-I |
| Solution of 1.0 wt. % alcohol of formulas (Ia) and/or | E-I** |
| (IIa)* in 1,2-propanediol | |

*active substance
**as contemplated herein

75 μL of the antiperspirant agent V-I and 75 μL of the cosmetic agent E-I were each applied to the backs of 16 female subjects, on either side next to the spine. After 5 minutes, the treated areas were covered with an occlusive non-adsorbent film. After 2 hours, these non-adsorbent pads were removed. The compositions were applied on four consecutive days to the backs of the female test subjects, each time in the above-described manner. 24 hours after the last application of the composition, absorbent pads were placed onto the areas on the backs of the female subjects where previously the compositions had been applied. Furthermore, pads were likewise applied to the other side of the spine at the same height, which served as controls. After the female subjects had been sweating in the sauna for approximately 15 minutes at 80° C., the amount of sweat absorbed by the pads was gravimetrically determined, wherein each composition was compared to the respectively corresponding untreated area on the back. Based on the gravimetric determination of the volume of sweat, the reduction in sweat was ascertained, wherein all ascertained values were statistically significant.

The reduction in sweat of the respective composition compared to an untreated skin area is shown in the table below.

| No. | Reduction in sweat |
|---|---|
| V-I | 4% |
| E-I | 17% |

The use of cosmetic agents comprising specific alcohols of formulas (Ia) and/or (Ib) results in a reduction in perspiration and reduced body odor when these agents are applied to the skin.

### 2. Formulations

The alcohol of formulas (I) and/or (II) used in the examples below is preferably an alcohol of formulas (Ia) and/or (IIa).

Antiperspirant suspension sticks as contemplated herein
(quantity information in wt. %)

| | 1.1 | 1.2 | 1.3 | 1.4 | 1.5 | 1.6 |
|---|---|---|---|---|---|---|
| Hydrogenated castor oil | — | — | — | 1.5 | 1.5 | 1.5 |
| Stearyl alcohol | 24.0 | 24.0 | 35.0 | 30.0 | 18 | 18 |
| Novata AB | — | — | — | 4 | 4 | 4 |
| Alcohol of formula (I) | 0.5 | 2 | — | 5 | 3 | — |
| Symsitive 1609 | — | — | 2 | — | — | 3 |
| PPG-14 butyl ether | 10.0 | 10.0 | 10.0 | 15.3 | 15.3 | 15.3 |
| Hydrogenated castor oil | 3.0 | 3.0 | 5.0 | — | — | — |
| Myristyl myristate | 1.5 | 2.0 | 1.5 | — | — | — |
| Performalene 400 | — | 1.5 | — | — | — | — |
| DL-Menthol | 0.2 | 0.2 | 0.2 | — | — | — |
| Eucalyptol | 0.2 | 0.2 | 0.2 | — | — | — |
| Anethole | 0.2 | 0.2 | 0.2 | — | — | — |
| Silica dimethyl silylate | 1.4 | 1.4 | 1.4 | — | — | — |
| Silica | 0.3 | 0.3 | 0.3 | — | — | — |
| Talcum | — | — | — | 3 | 3 | 3 |
| Emulgin B1 | — | — | — | 3 | 3 | 3 |
| Perfume | 2.0 | 2.0 | 2.0 | 1 | 1 | 1 |
| Cyclomethicone (at least | to | to | to | to | to | to |
| 95 wt. % cyclopentasiloxane) | make | make | make | make | make | make |
| | 100 | 100 | 100 | 100 | 100 | 100 |

Antiperspirant suspension sticks as contemplated herein
(quantity information in wt. %)

| | 2.1 | 2.2 | 2.3 | 2.4 | 2.5 | 2.6 |
|---|---|---|---|---|---|---|
| Hydrogenated castor oil | — | — | — | 1.5 | 1.5 | 1.5 |
| Stearyl alcohol | 24.0 | 24.0 | 30.0 | 30 | 18 | 18 |
| Novata AB | — | — | — | 4 | 4 | 4 |
| Alcohol of formula (I) | 0.5 | 2 | — | 5 | 3 | — |
| Symsitive 1609 | — | — | 2 | — | — | 3 |
| PPG-14 butyl ether | 10.0 | 10.0 | 10.0 | 15.3 | 15.3 | 15.3 |
| Hydrogenated castor oil | 3.0 | 3.0 | 5.0 | — | — | — |
| Myristyl myristate | 1.5 | 1.5 | 1.5 | — | — | — |
| Performalene 400 | — | 1.5 | — | — | — | — |
| DL-Menthol | 0.2 | 0.2 | 0.2 | — | — | — |
| Eucalyptol | 0.2 | 0.2 | 0.2 | — | — | — |
| Anethole | 0.2 | 0.2 | 0.2 | — | — | — |
| Silica dimethyl silylate | 1.4 | 1.4 | 1.4 | — | — | — |
| Silica | 0.3 | 0.3 | 0.3 | — | — | — |
| Talcum | — | — | — | 3 | 3 | 3 |
| Emulgin B1 | — | — | — | 3 | 3 | 3 |
| Perfume | 2.0 | 2.0 | 2.0 | 1 | 1 | 1 |
| Cyclomethicone (at least | to | to | to | to | to | to |
| 95 wt. % cyclopentasiloxane) | make | make | make | make | make | make |
| | 100 | 100 | 100 | 100 | 100 | 100 |

Antiperspirant sticks as contemplated herein in the form of an
oil-in-water emulsion (quantity information in wt. %)

| | 3.1 | 3.2 | 3.3 |
|---|---|---|---|
| Cutina ® AGS | 2.5 | 2.5 | 2.5 |
| Cutina ® FS45 | 3.5 | 3.5 | 3.5 |
| Eumulgin ® B2 | 0.8 | 0.8 | 0.8 |
| Eumulgin ® B3 | 0.8 | 0.8 | 0.8 |
| Diisopropyl adipate | 6.0 | 6.0 | 6.0 |
| Novata ® AB | 4.0 | 4.0 | 4.0 |
| Cutina ® CP | 5.0 | 5.0 | 5.0 |
| Cutina ® HR | 4.0 | 4.0 | 4.0 |
| Kester Wax K62 | 5.0 | 5.0 | 5.0 |
| Talkum Pharma G | 10 | 10 | 10 |
| Perfume | 1.2 | 1.2 | 1.2 |
| 2-benzylheptane-1-ol | — | 0.3 | 0.3 |
| Sensiva SC 50 | 0.6 | 0.6 | 0.6 |
| 1,2-propanediol | 10 | 10 | 10 |
| Alcohol of formula (I) | 0.5 | 2 | — |
| Symsitive 1609 | — | — | 5 |
| Water, deionized | to make 100 | to make 100 | to make 100 |

Antiperspirant microemulsions (information in wt. %)

| | 4.1 | 4.2 | 4.3 | 4.4 |
|---|---|---|---|---|
| Plantaren ® 1200 | 1.7 | 1.7 | — | — |
| Plantaren ® 2000 | 1.1 | 1.4 | 2.4 | 2.4 |
| Glycerol monooleate | 0.71 | 0.71 | — | — |
| Dioctyl ether | 4.0 | 4.0 | 0.090 | 0.090 |
| Octyldodecanol | 1.0 | 1.0 | 0.020 | 0.020 |
| Perfume oil | 1.0 | 1.0 | 1.0 | 1.0 |
| 1,2-propylene glycol | 5.0 | 5.0 | — | — |
| Glycerol | — | — | 5.0 | 5.0 |
| 2-benzylheptane-1-ol | 0.50 | — | — | — |
| Triethyl citrate | — | 0.50 | 0.50 | 0.50 |
| Triclosan | 0.10 | — | — | — |
| Alcohol of formula (I) | 0.5 | 2 | — | 5 |
| Symsitive 1609 | — | — | 2 | — |
| Water | to make | to make | to make | to make |
| | 100 | 100 | 100 | 100 |

Antiperspirant roll-ons (quantity information in wt. %)

| | 5.1 | 5.2 | 5.3 | 5.4 |
|---|---|---|---|---|
| Ethanol 96% (DEP denatured) | 30 | 30 | 28 | 28 |
| Mergital ® CS 11 | 2.0 | 2.0 | — | — |
| Eumulgin ® B3 | 2.0 | 2.0 | 2.0 | 2.0 |
| Emulgin ® B1 | — | — | 2.0 | 2.0 |
| Hydroxyethyl cellulose | 0.50 | 0.50 | 0.30 | 0.30 |
| Alcohol of formula (I) | 0.5 | 2 | — | 5 |
| Symsitive 1609 | — | — | 2 | — |
| Cocamidopropyl PG dimonium | 0.20 | — | — | — |
| chloride phosphate | | | | |
| Perfume oil | 0.80 | 0.80 | 1.0 | 1.0 |
| Water | to make | to make | to make | to make |
| | 100 | 100 | 100 | 100 |

Spray suspension (quantity information in wt. %, based
on the total weight of the propellant-free composition)

| | 6.1 | 6.2 | 6.3 | 6.4 | 6.5 | 6.6 | 6.7 | 6.8 |
|---|---|---|---|---|---|---|---|---|
| Bentone 38 V CG | 5.00 | 3.93 | 4.00 | 5.00 | 5.00 | 3.93 | 4.00 | 5.00 |
| Propylene carbonate | 1.50 | 0.71 | 1.50 | 1.80 | 1.50 | 0.71 | 1.50 | 1.80 |
| Fragrance | 7.14 | 6.50 | 5.00 | 6.50 | 7.14 | 6.50 | 5.00 | 6.50 |
| 2-Ethylhexyl palmitate | — | 73.57 | 25.0 | — | — | 73.57 | — | — |
| Abil K 4 | 48.4 | — | — | — | — | — | — | — |
| Alcohol of formula (I) | 0.5 | 2 | — | 5 | 0.5 | 2 | — | 5 |
| Symsitive 1609 | — | — | 2 | — | — | — | 2 | — |
| Finsolv TN | — | — | — | — | 15.0 | — | — | — |
| Ucon Fluid AP | — | — | — | — | — | — | 12.0 | 10.0 |
| Dimethicone 5 Cst | — | — | — | — | — | — | — | 5.0 |
| Isopropyl myristate | 7.37 | — | 10.00 | 10.00 | — | — | — | — |
| Triethyl citrate | — | — | 10.5 | 12.00 | — | — | 10.5 | 12.00 |
| C10-C13 isoalkane | — | — | — | 20.0 | — | — | | 3.0 |
| Cyclopentasiloxane | to | to | to | to | to | to | to | to |
| | make | make | make | make | make | make | make | make |
| | 100 | 100 | 100 | 100 | 100 | 100 | 100 | 100 |

Exemplary compositions 6.1 to 6.8 were bottled in an aluminum spray can, optionally coated with epoxy-phenolic lacquer, at a weight ratio of the propellant (butane/propane/isobutane mixture) to the suspension of 80:20 and 85:15 and 60:40 and 90:10.

Spray emulsion (quantity information in wt. %, based on the
total weight of the propellant-free composition)

| | 7.1 | 7.2 | 7.3 |
|---|---|---|---|
| C₁₀-C₁₃ isoalkane | 8.90 | — | 8.90 |
| Cyclopentasiloxane | — | 8.90 | — |
| Dow Corning ES-5227 DM | 1.40 | 1.40 | 1.40 |
| Isoceteth-20 | 0.500 | 0.500 | 0.500 |
| Dimethicone | 4.20 | 4.20 | 4.20 |
| Isopropyl myristate | 9.00 | 9.00 | 9.00 |
| 1,2-propanediol | 7.00 | 25.0 | 25.0 |
| Phenoxyethanol | 0.500 | 0.500 | 0.500 |
| Perfume | 2.50 | 2.50 | 2.50 |
| Alcohol of formula (I) | 0.500 | 3.00 | — |
| Symsitive 1609 | — | — | 2.00 |
| L-menthol | 0.400 | 0.300 | — |
| trans-anethole | — | 0.300 | — |
| Eucalyptol | — | 0.300 | — |
| Water | to make 100 | to make 100 | to make 100 |

Exemplary compositions 7.1 to 7.3 were bottled in an aluminum spray can, optionally coated with epoxy-phenolic lacquer, at a weight ratio of the propellant (butane/propane/isobutane mixture) to the spray emulsion of 80:20 and 85:15 and 60:40 and 90:10.

Antiperspirant cosmetic agents as contemplated herein
(quantity information in wt. %, based on the total
weight of the propellant-free composition)

| | 8.1 | 8.2 | 8.3 | 8.4 |
|---|---|---|---|---|
| Cyclomethicone D5 | to make | to make | to make | to make |
| | 100 | 100 | 100 | 100 |
| Bentone 38 V CG | 5.00 | 3.93 | — | 5.00 |
| Propylene carbonate | 1.50 | 0.71 | — | 1.80 |
| Fragrance | 7.14 | 6.50 | 5.00 | 6.50 |
| 2-Ethylhexyl palmitate | — | 25 | — | — |
| Isopropyl myristate | 7.37 | — | 10.00 | 12.0 |
| Triethyl citrate | — | — | 10.5 | 12.0 |
| Dimethicone 2-10 cst | — | — | — | 10.0 |
| C10-C13 isoalkane | — | 10.0 | 35.39 | — |
| Alcohol of formula (I) | 0.500 | 2.00 | — | 5.00 |
| Symsitive 1609 | — | — | 2.00 | — |

Exemplary compositions 8.1 to 8.4 were bottled in an aluminum spray can, optionally coated with epoxy-phenolic lacquer, at a weight ratio of the propellant (butane/propane/isobutane mixture) to the suspension of 80:20 and 85:15 and 60:40 and 90:10.

Roll-on O/W emulsion (quantity information in wt. %)

| | 9.1 | 9.2 | 9.3 |
|---|---|---|---|
| Potassium aluminum sulfate | 1.50 | — | — |
| KAI(SO₄)₂•12H₂O | | | |
| Talc | 1.0 | — | — |
| Bentonite | — | 1.00 | — |
| Hectorite | — | — | — |
| Brij S 2 | 2.50 | 2.50 | 2.50 |
| Brij S 721 | 1.50 | 1.50 | 1.50 |
| Perfume | 1.10 | 1.10 | 1.10 |
| Arlamol E | 0.500 | 0.500 | 0.500 |
| Bisabolol | 0.100 | 0.100 | 0.100 |
| Dry Flo PC | 0.100 | 0.100 | 0.100 |
| Alcohol of formula (I) | 0.500 | 2.00 | — |
| Symsitive 1609 | — | — | 4.00 |
| Dow Corning 2501 Cosmetic | 0.100 | 0.100 | 0.100 |
| Wax | | | |
| Tocopheryl acetate | 0.100 | 0.100 | 0.100 |
| Water | to make 100 | to make 100 | to make 100 |

Stick-shaped W/O emulsions (quantity information in wt. %)

| | 10.1 | 10.2 | 10.3 | 10.4 |
|---|---|---|---|---|
| Propylene glycol | 5.00 | 5.00 | 7.50 | 9.50 |
| C12-C15 alkyl benzoate | 8.04 | 8.04 | 8.04 | 8.04 |
| Dimethicone 2 cst | 6.43 | 6.43 | 6.43 | 6.43 |
| Dimethicone 5 cst | 1.57 | 1.57 | 1.57 | 1.57 |
| polyethylene | 10.2 | 11.7 | 9.70 | 12.2 |
| Abil EM 90 | 0.998 | 0.998 | 0.998 | 0.998 |
| Abil EM 97 | 1.22 | 1.22 | 1.22 | 1.22 |
| 4-tert-Butylcyclohexanol | 0.500 | 2.00 | — | 5.00 |
| Symsitive 1609 | — | — | 2.00 | — |
| Synthetic wax | 0.100 | 0.100 | 0.100 | 0.100 |
| Perfume | 1.50 | 1.50 | 1.20 | 1.50 |
| Water | to make | to make | to make | to make |
| | 100 | 100 | 100 | 100 |

Anhydrous suspension roll-on (quantity information in wt. %)

| | 11.1 | 11.2 | 11.3 | 11.4 |
|---|---|---|---|---|
| Cyclopentasiloxane | to make 100 | to make | to make | to make |
| | | 100 | 100 | 100 |
| Bentone 38 V (hydrophobic) | 2.00 | 3.00 | — | — |
| Propylene carbonate | 1.50 | 2.00 | — | — |
| Ethylhexyl palmitate | 20.0 | 10.0 | — | — |
| Finsolv TN | — | 10.0 | 20.0 | 10.0 |
| Aerosil 972 V (hydrophobic) | — | — | 3.00 | 1.00 |
| Aerosil 200 | — | — | — | 1.00 |
| Protectate MOD 3 | — | 0.500 | — | 0.300 |
| Alcohol of formula (I) | 0.500 | 2.00 | — | 5.00 |
| Symsitive 1609 | — | — | 3.00 | — |
| Perfume | 1.00 | 1.00 | 2.00 | 2.00 |

Anhydrous deodorant sprays (information in wt. %)

| | 12.1 | 12.2 | 12.3 | 12.4 | 12.5 |
|---|---|---|---|---|---|
| Ethanol (100%) | 22.0 | 22.0 | 22.0 | 22.0 | 22.0 |
| Alcohol of formula (I) | 0.500 | 5.00 | 3.00 | — | — |
| Symsitive 1609 | — | — | — | 1.00 | 7.00 |
| Symdiol 68 T | — | — | 1.0 | — | — |
| Triethyl citrate | 3.0 | 1.5 | 1.5 | 1.5 | 1.5 |
| Cocamidopropyl PG | 0.3 | 0.1 | 0.1 | 0.1 | 0.1 |
| dimonium chloride | | | | | |
| phosphate | | | | | |
| 2-Ethylhexyl glycerol | 0.3 | 0.3 | 0.3 | 0.3 | 0.3 |
| 2-benzylheptanol | 0.1 | 0.1 | 0.1 | 0.1 | 0.1 |
| Benzyl alcohol | 0.1 | 0.1 | 0.1 | 0.1 | 0.1 |
| Phenoxyethanol | 0.4 | 0.4 | 0.4 | 0.4 | 0.4 |
| Perfume | 0.7 | 0.7 | 0.7 | 0.7 | 0.7 |
| Diethyl phthalate | 0.1 | 0.1 | 0.1 | 0.1 | 0.1 |
| Propane | 9.0 | 9.0 | 9.0 | 9.0 | 9.0 |
| Isobutane | 2.0 | 2.0 | 2.0 | 2.0 | 2.0 |
| Butane | to make | to make | to make | to make | to make |
| | 100.0 | 100.0 | 100.0 | 100.0 | 100.0 |

Deodorant in pump atomizer (information in wt. %)

| | 13.1 | 13.2 |
|---|---|---|
| Ethanol 96% (DEP denatured) | 55.0 | 75.0 |
| Triethyl citrate | 3.0 | 3.0 |
| Cremophor ® RH 455 | 0.5 | 0.5 |
| Eucarol ® AGE/EC/UP | 1.0 | 1.0 |
| Alcohol of formula (I) | 1.5 | — |
| Symsitive 1609 | — | 3.0 |
| Cocamidopropyl PG dimonium chloride phosphate | 0.4 | 0.4 |
| Perfume oil | 1.0 | 1.0 |
| Water | to make | to make |
| | 100 | 100 |

Soap-containing deodorant sticks (information in wt. %)

| | 14.1 | 14.2 | 14.3 | 14.4 | 14.5 |
|---|---|---|---|---|---|
| Cutina ® FS 45 | 4.4 | 4.4 | 4.4 | 4.4 | 4.4 |
| 1,3-butanediol | 31.7 | 31.7 | 31.7 | 31.7 | |
| 1,2-propylene glycol | 21.0 | 21.0 | 21.0 | 21.0 | 21.0 |
| Glycerol | | | | | 30 |
| Octyldodecanol | 2.0 | 2.0 | 2.0 | 2.0 | 2.0 |
| PPG-5 laureth-5 | 3.0 | 3.0 | 3.0 | 3.0 | 3.0 |
| PEG-40 Hydrogenated | 0.05 | 0.05 | 0.05 | 0.05 | 0.05 |
| Castor Oil | | | | | |
| NaOH 45% | 1.44 | 1.44 | 1.44 | 1.44 | 1.44 |
| Phenoxyethanol | 1.0 | 1.0 | 1.0 | 1.0 | 1.0 |
| Sensiva ® SC 50 | 0.3 | 0.3 | 0.3 | 0.3 | 0.3 |
| Perfume oil | 2.0 | 2.0 | 2.0 | 2.0 | 2.0 |
| Alcohol of formula (I) | 1.5 | — | 0.5 | — | 5 |
| Symsitive 1609 | — | 4 | — | 2 | — |
| 2-benzylheptane-1-ol | 0.3 | — | — | — | — |
| Anise alcohol | — | 0.3 | — | 0.3 | — |
| 2-methyl-5-phenyl- | — | — | 0.3 | — | 0.3 |
| pentane-1-ol | | | | | |
| Ethanol | to make | to make | to make | to make | to make |
| | 100 | 100 | 100 | 100 | 100 |

Alcoholic deodorant Spray (quantity information in wt. %, based
on the total weight of the propellant-free composition)

| | 15.1 | 15.2 |
|---|---|---|
| Ethanol 96% (DEP denatured) | to make 100 | to make 100 |
| Triethyl citrate | 3.0 | 3.0 |
| PEG-40 Hydrogenated Castor Oil | 0.5 | 0.5 |
| Plantaren 1200 | 1.0 | 3.0 |
| Alcohol of formula (I) | 1.5 | — |
| Symsitive 1609 | — | 6.0 |
| Cocamidopropyl PG dimonium | 0.4 | 0.4 |
| chloride phosphate | | |
| Perfume oil | 1.0 | 1.0 |
| Water | 4 | 0.1 |

90 to 99.9 wt. % of the exemplary compositions 15.1 and 15.2 were bottled in an aluminum spray can, optionally coated with epoxy-phenolic lacquer. The necessary pressure for the application was built using <10% compressed air, nitrogen, nitrous oxide, carbon dioxide, or other compressible gases.

The following commercial products were used:

| Commercial product | INCI | Supplier/manufacturer |
|---|---|---|
| Abil EM 90 | CETYL PEG/PPG-10/1 dimethicone | Evonik |
| Abil EM 97 | Bis-PEG/PPG-14/14 dimethicone, | Evonik |
| | cyclomethicone | |
| Abil K 4 | Cyclomethicone | Goldschmidt |
| Arlamol E | PPG-15 Stearyl ether | Croda |
| Bentone 38 V CG | Disteardimonium hectorite | Elementis Specialities |
| Brij IC 20 | Isoceteth-20 | Croda |
| Brij S 2 | Steareth-2 | Croda |
| Brij S 721 | Steareth-21 | Croda |
| Cutina ® CP | Cetyl palmitate | BASF |
| Cutina ® FS45 | Palmitic acid, stearic acid | BASF |
| Cutina ® HR | Hydrogenated castor oil | BASF |
| Dow Corning ® 245 | Cyclopentasiloxane | Dow Corning |
| Dow Corning ® 2501 | Bis-PEG-18 Methyl ether dimethyl silane | Dow Corning |
| Dow Corning ES-5227 DM | Dimethicone, PEG/PPG-18/18 dimethicone | Dow Corning |
| | at a weight ratio of 3:1 | |
| Dry Flo PC | Aluminum starch octenylsuccinate | National Starch |
| Eumulgin ® B1 | Ceteareth-12 | BASF |
| Eumulgin ® B2 | Ceteareth-20 | BASF |
| Eumulgin ® B3 | Ceteareth-30 | BASF |
| Finsolv TN | C12-15 alkyl benzoate | Innospec Performance |
| | | Chemicals |
| Kester Wax K62 | Cetearyl behenate | Koster Keunen |
| Finsolv TN | C12-15 Alkyl benzoate | Innospec |
| Locron L (AS = 50%) | Aluminum chlorohydrate | Clariant |
| Mergital ® CS 11 | Ceteareth-11 | BASF |
| Novata ® AB | Cocoglycerides (melting point 30-32° C.) | BASF |
| Performalene 400 | Polyethylene | New Phase Technology |
| Plantaren ® 1200 | LAURYL GLUCOSIDE, approx. 50% AS | BASF |
| Plantaren ® 2000 | DECYL GLUCOSIDE, approx.. 50% AS | BASF |
| Sensiva ® SC 50 | 2-ethylhexyl glycerol ether | Schülke & Mayr |
| Symdiol 68 T | Hexanediol, Caprylyl Glycol, Tropolone | Symrise |
| Symsitive 1609 | Pentylene glycol, 4-tert-Butylcyclohexanol | Symrise |
| Ucon Fluid AP | PPG-14 butyl ether | Dow Chemical |

While at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing an exemplary embodiment of the disclosure. It being understood that various changes may be made in the function and arrangement of elements described in an exemplary embodiment without departing from the scope of the disclosure as set forth in the appended claims.

1. A cosmetic method for reducing perspiration of the body and/or for reducing body odor triggered by perspiration, the method comprising the steps of:
applying an antiperspirant cosmetic agent (M1) to human skin, wherein the antiperspirant cosmetic agent (M1), in a cosmetically compatible carrier, comprises, based on the total weight of the cosmetic agent (M1),
a) about 0.1 to about 5.0 wt. % of at least one alcohol of formulas (I) and/or (II) 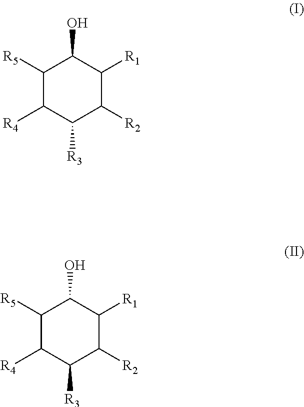
wherein
R₁ to R₅, each independently of one another, denote hydrogen, a linear or branched C₁ to C₁₀ alkyl group or a linear or branched C₂ to C₁₀ alkylene group; and
b) no more than 1 wt. % antiperspirant aluminum salts and/or aluminum-zirconium salts; and
maintaining the antiperspirant cosmetic agent (M1) on the application site for at least an hour.

2. The method according to claim 1, wherein the groups R₁, R₂, R₄ and R₅ in formulas (I) and/or (II), each independently of one another, denote hydrogen.

3. The method according to claim 1, wherein the group R₃ in formulas (I) and/or (II) denotes a branched C₃ to C₈ alkyl group.

4. The method according to claim 1, wherein the antiperspirant cosmetic agent (M1) comprises at least one alcohol of formulas (Ia) and/or (IIa)

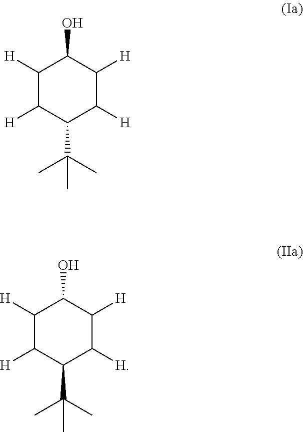

5. The method according to claim 1, wherein the cosmetic agent (M1), based on the total weight thereof, comprises about 0.1 to about 5.0 wt. % of at least one alcohol of formulas (I) and/or (II).

6. The method according to claim 1, wherein the cosmetic agent (M1) comprises 0 wt. % of antiperspirant aluminum salts and/or aluminum-zirconium salts, based on the total weight of the cosmetic agent (M1).

7. The method according to claim 1, wherein the cosmetic agent (M1) has a pH value of about pH 2 to about pH 10.

8. The method according to claim 1, further comprising the step of applying a cosmetic agent (M2) to the skin, wherein the cosmetic agent (M2) comprises at least one antiperspirant aluminum salt and/or aluminum zirconium salt in a cosmetically compatible carrier.

9. A packaging unit, comprising,
formulated separately from one another,
a) at least one first container (C1), containing a cosmetic agent (M1) comprising, in a cosmetically compatible carrier, at least one alcohol of formulas (I) and/or (II) 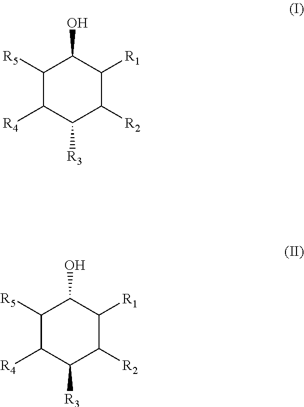
wherein
R₁ to R₅, each independently of one another, denote hydrogen, a linear or branched C₁ to C₁₀ alkyl group or a linear or branched C₂ to C₁₀ alkylene group; and
no more than 1 wt. % antiperspirant aluminum salts and/or aluminum-zirconium salts, based on the total weight of the cosmetic agent (M1), and
b) at least one second container (C2), containing a cosmetic agent (M2) comprising at least one antiperspirant active ingredient.

10. The packaging unit according to claim 9, wherein the cosmetic agent (M1) comprises at least one alcohol of formulas (Ia) and/or (IIa)

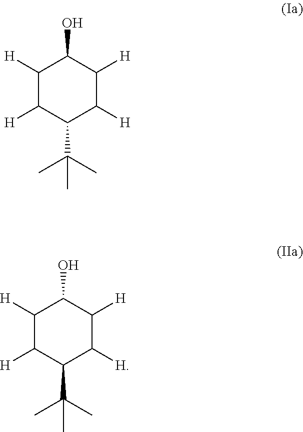

11. (canceled)

12. (canceled)

13. The method according to claim 1 wherein the group R₃ in formulas (I) and/or (II) denotes a branched C₃ to C₇ alkyl group.

14. The method according to claim 1 wherein the group R₃ in formulas (I) and/or (II) denotes a branched C₃ to C₅ alkyl group.

15. The method according to claim 1, wherein the cosmetic agent (M1), based on the total weight thereof, comprises about 0.6 to about 3.0 wt. % of at least one alcohol of formulas (I) and/or (II).

16. The method according to claim 1, wherein the cosmetic agent (M1), based on the total weight thereof, comprises about 0.7 to about 2.0 wt. % of at least one alcohol of formulas (I) and/or (II).

17. The method according to claim 1, wherein the cosmetic agent (M1), based on the total weight thereof, comprises about 0.8 to about 1.5 wt. % of at least one alcohol of formulas (I) and/or (II)

18. The method according to claim 4, wherein the cosmetic agent (M1), based on the total weight thereof, comprises about 0.1 to about 5.0 wt. % of at least one alcohol of formulas (Ia) and/or (Ib).

19. The method according to claim 4, wherein the cosmetic agent (M1), based on the total weight thereof, comprises about 0.6 to about 3.0 wt. % of at least one alcohol of formulas (Ia) and/or (Ib).

20. The method according to claim 4, wherein the cosmetic agent (M1), based on the total weight thereof, comprises about 0.7 to about 2.0 wt. % of at least one alcohol of formulas (Ia) and/or (Ib).

21. The method according to claim 4, wherein the cosmetic agent (M1), based on the total weight thereof, comprises about 0.8 to about 1.5 wt. % of at least one alcohol of formulas (Ia) and/or (Ib).

22. The packaging unit according to claim 9, wherein the group R₃ in formulas (I) and/or (II) denotes a branched C₃ to C₇ alkyl group.
